(19) United States
(12) Patent Application Publication
(10) Pub. No.: US 2004/0023262 A1
(43) Pub. Date: Feb. 5, 2004

(54) Targeted manipulation of genes in plants

(75) Inventors: Pramod B. Mahajan, Urbandale, IA (US); Priya Kannan, Ankeny, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/377,972

(22) Filed: Feb. 28, 2003

Related U.S. Application Data
(60) Provisional application No. 60/361,798, filed on Mar. 5, 2002.

(51) Int. Cl. C12Q 1/68; C07H 21/04; A01H 1/00; C12N 15/82

(52) U.S. Cl. 435/6; 800/286; 536/23.2

(57) ABSTRACT

Methods and compositions for altering a polynucleotide of interest in a plant are provided. The methods involve introducing a template oligonucleotide into a plant cell, the template oligonucleotide comprises regions complementary to the plant polynucleotide, the complementary regions flank a non-complementary region, and the template oligonucleotide comprises a single-stranded nucleic acid comprising at least one cationic phosphoramidate internucleoside linkage.

This application claims priority to U.S. Serial No. 60/361,798 filed Mar. 5, 2002 the disclosure of which is incorporated herein by reference.

### FIELD OF INVENTION

The present invention is drawn to the genetic modification of higher plants.

### BACKGROUND OF THE INVENTION

Because of the complex organization and metabolic compartmentation in higher plants, many molecular tools have to be combined to successfully genetically manipulate a plant. At the present time, directed changes require a transformation system, a suitable gene, a promoter sequence capable of driving expression of the gene, effective targeting signals to direct the expression product to the correct destination in the cell, and often other regulatory elements.

There are many published systems for the transformation of plant cells. However, there remain drawbacks to each of the systems. For example, Agrobacterium tumefaciens gene transfer is widely used for creating transgenic plants, however, in some plants making a successful transformation utilizing the Agrobacterium system is difficult. Other systems include particle bombardment, viral vectors, protoplast transformation via polyethylene glycol or electroporation, microinjection of DNA into protoplast, and macroinjection of DNA. These transformation systems suffer from a lack of genomic targeting control for insertion of the foreign gene. In addition, bombardment results in multiple insertions of the foreign gene into the plant genome.

Any transformation system in plants must deal with the problems of cosuppression which occurs when endogenous genes are down-regulated by expression of homologous sense transcripts. Expression of gene constructs have led to the down-regulation of genes in transgenic plants, however the effects of cosuppression are variable, and the underlying mechanisms remain unclear.

Site-directed manipulation of chromosomal genes has become the method of choice for determining gene function and bacterial, yeast, and mammalian cells. The primary methods used in site-directed gene manipulation rely on gene replacement via homologous recombination using an appropriately designed gene targeting vector. In plants, gene targeting has been limited by the frequency of homologous recombination. Even with improvements in transformation and selection methods, the frequency of gene targeting in plants is still 10⁻³-10⁻⁴ fold lower than random integration.

Herbicide resistant forms of plants are desirable for many breeding and crop production applications. Approaches to date have involved laborious methods including: finding a naturally existing form of resistance in a plant and introgressing the trait into desirable germplasm; mutagenesis of plants, seeds, and seedlings to generate novel mutant plants that confer resistance and introgressing the trait into the breeding population; finding a naturally existing form of a gene which confers resistance to a target herbicide and introducing the gene into the desired species by transformation; and, converting a wild type gene to a resistant form by mutagenesis. All of these approaches rely on either natural recovery of the trait or modification of the gene and subsequent introduction of the resistance gene into a plant.

A major disadvantage in each of these approaches is the time involved in terms of mutagenesis, recovery of the trait and the breeding necessary to introduce the trait into desired populations. Further, where transformation is involved, plants will have to be tested and selected that are not impacted by expression instability or by poor agronomic performance. Additionally, in many instances, optimum performance of a gene in a given species may only be achieved following resynthesis of the gene to maximize usage of preferred codons, or by the creation of modified forms of the gene.

Because of the present problems associated with the integration and expression of foreign genes in plant cells, effective strategies for modification, conversion or correction of gene sequences are needed.

### SUMMARY OF THE INVENTION

Compositions and methods for modifying DNA sequences of interest in a plant cell are provided. The methods comprise introducing a template oligonucleotide, regions of which are complementary to a plant genomic DNA of interest. As used herein plant genomic DNA includes nuclear and organellar DNA. Genomic DNA also includes DNA transformed into the plant genome, transformed DNA. The complementary regions flank at least one non-complementary base pair that replaces the naturally occurring sequence in the plant genome. The template oligonucleotide comprises a single-stranded DNA comprising ethylenediamine phosphoramidate internucleoside linkages.

### DETAILED DESCRIPTION OF THE INVENTION

The template oligonucleotides of the invention comprise oligonucleotides having regions of homology to the genomic DNA of interest in the plant and at least one region of non-homology. The template oligonucleotides of the invention are typically single-stranded DNA. Generally the template oligonucleotide will be less than about 200 nucleotides, typically about 20 to about 80 nucleotides, or about 20 to about 60 nucleotides in size. The regions of homology will generally be at least 5 nucleotides in length. The region of non-homology will comprise at least one nucleotide. The region of non-homology will generally be up to 5 nucleotides in length, or up to 10 nucleotides in length. The template oligonucleotide may have polyT regions near the ends of the template. In selecting and preparing the template oligonucleotide, the complementary regions can be complementary to either a transcribed or a non-transcribed strand of the plant genomic DNA.

A portion of the template oligonucleotide will comprise cationic phosphoramidate internucleoside linkages (cationic oligonucleotides). Methods for preparing cationic oligonucleotides having such linkages are found in Dagle & Weeks, Nucl. Acids Res. 24:2143-2149, 1996; Dagle et al., Nucl. Acids Res 28:2153-2157, 2000; U.S. Pat. No. 6,274,313 and U.S. Pat. No. 5,734,040 the disclosures of which are incorporated herein by reference. The cationic phosphoramidate is incorporated during oligonucleotide synthesis via oxidative amidation. In one embodiment the cationic phosphoramidate have both a primary and a tertiary amine. Positively charged internucleoside linkages can be generated by using a diethylethylenediamine such as N,N-diethylethylenediamine (DEED) and methoxyehtylamine phosphoramidates. The resulting cationic oligonucleotides will have improved stability in eukaryotes. The improved stability is useful in targeted genomic modifications.

Typically the cationic oligonucleotide will comprise the ends of the template oligonucleotide, however a large portion or even the entire length of the template oligonucleotide can comprise cationic oligonucleotides. The cationic oligonucleotides add stability to the template oligonucleotide. The template oligonucleotide will comprise cationic oligonucleotides in an amount sufficient to provide stability to the template oligonucleotide from endonucleases. Typically the cationic oligonucleotide portion is at least about 5 nucleotides in length, at least about 10 nucleotides in length, or at least about 15 nucleotides in length. Generally the cationic oligonucleotide portion will comprise at least about 10%, 20%, 30%, 40%, 50%, 60%, 70%, 80%, 90% of the template oligonucleotide and up to the entire length of the template oligonucleotide.

It is further recognized that where more than one point mutation is being inserted into the genome, that the template oligonucleotide can be constructed to have more than two flanking complementary sequences. That is, complementary sequences may flank more than one modifying DNA sequence (non-complementary region). Such a design may provide better stability and recombination efficiency where the sites of mutation or modification are not contiguous.

The template oligonucleotides of the invention are intended to specifically introduce alterations into target genomic DNA. The template oligonucleotides are designed to have at least two complementary regions flanking an interposed non-complementary region. The non-complementary region contains the “mutator” base pairs or sequence to be introduced into the target. In this manner, a predetermined alteration can be made in the target sequence.

Methods for construction of the non-cationic portion (if present) of the template oligonucleotides of the invention are known in the art. The oligonucleotides can be synthesized by solid phase synthesis. See, Caruthers, M. H. (1985) Science 230:281-285; Itakura et al. (1984) Ann. Rev. Biochem. 53:523-556. See, for example, Scaringe et al. (1990) Nucleic Acids Research 18:5433-5441; Usman et al. (1992) Nucleic Acids Research 20:6695-6699; Swiderski et al. (1994) Anal. Biochem. 216:83-88; Usman and Cedergren (1992) Trends Biochem. Sci. 17:334-339; Yoon et al. (1996) Proc. Natl. Acad. Sci. USA 93:2071-2076; Cole-Strauss et al. (1996) Science 273:1386-1389; and Kren et al. (1997) Hepatology 25:1462-1468. Such disclosures are herein incorporated by reference.

The compositions and methods of the invention are useful for targeted gene correction, site-specific mutagenesis, gene knockout, allelic replacement and genetic modification of plant genomes. Furthermore, the methods can be used to create a predetermined nucleotide pair mismatch in a target sequence of the genome of a plant or plant cell upon which endogenous mismatch repair mechanisms can operate to create a nucleotide alteration at or near the target sequence. The methods can be used to produce a nucleotide deletion. The methods are useful for modulating the activity of genes of interest.

In this manner, the template oligonucleotides of the invention can be used to introduce a modification in a specific genomic location in a plant cell. The specific location of the modification is defined by the nucleic acid sequence called the target sequence. The change to be introduced is encoded by the non-complementary region. As indicated above, the modification may be in substituting one or more than one bases of the sequence, adding one or more bases, or deleting one or more bases in the native gene. The modification can be in the regulatory region of a gene, in the promoter region, in the 3′ or 5′ non-translated region of a gene, in the coding region of a gene, in the junction between an intron and an exon, or in a transformed region, i.e. non-native DNA. For example a stop codon can be inserted in a coding region to prevent expression of a protein.

The methods are useful for inactivating or altering a gene or a DNA sequence of interest. The gene can be an endogenous gene or a transposon or a gene that has been introduced into the plant genome by transformation methods. Genes of particular interest include genes that confer herbicide tolerance, scorable and/or selectable marker genes, genes regulating oil quantity and profile, genes regulating amino acid levels, genes altering starch properties, recombinase genes, genes affecting the nutrition and flavor properties, or genes in the metabolic pathways influenced by antioxidants or genes involved in plant development, differentiation, or maturity, genes involved in flower, seed and fruit development or ripening and the like. The method can also be used to alter recombinase sites.

As noted above, the methods and compositions can be used for generating herbicide resistant plants. The compositions comprise single strand oligonucleotides that comprise regions complementary to a plant genomic DNA of interest flanking base pairs necessary to convert the sequence to a herbicide resistant form of the gene. The method involves converting the naturally occurring nucleotide sequence in a plant by targeted gene conversion to create a herbicide resistance form of the gene. Herbicide resistant plants can be obtained from the method. Herbicide resistant plants can be created by modifying existing genes within the plant genome. Such genes include the 5-enol pyruvylshikimate-3-phosphate synthase (EPSPS) gene, the acetohydroxy acid synthase gene (AHAS or ALS) (Zhu T, Peterson DJ, Tagliani L, St Clair G, Baszczynski CL, Bowen B (1999) Proc Natl Acad Sci USA 96:8768-73, Zhu T, Mettenburg K, Peterson D J, Tagliani L, Baszczynski C L (2000) Nat Biotechnol 18:555-8) and the glyphosate N-acyl transferase gene (GAT) (WO 02/36782).

The template oligonucleotides can be introduced into the plant cell by any method available in the art. In this manner, genetically modified plants, plant cells, plant tissue, seed, and the like can be obtained. The template oligonucleotides may be introduced into the plant by one or more techniques typically used for direct DNA delivery into cells. Such protocols may vary depending on the type of plant or plant cell, i.e. monocot or dicot, targeted for gene modification. Suitable methods of transforming plant cells include microinjection (Crossway et al. (1986) Biotechniques 4:320-334), electroporation (Riggs et al. (1986) Proc. Natl. Acad. Sci. USA 83:5602-5606, direct gene transfer (Paszkowski et al. (1984) EMBO J. 3:2717-2722), and ballistic particle acceleration (see, for example, Sanford et al., U.S. Pat. No. 4,945,050; WO 91/10725 and McCabe et al. (1988) Biotechnology 6:923-926). Also see, Weissinger et al. (1988) Annual Rev. Genet. 22:421-477; Sanford et al. (1987) Particulate Science and Technology 5:27-37 (onion); Christou et al. (1988) Plant Physiol. 87:671-674 (soybean); McCabe et al. (1988) Biotechnology 6:923-926 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); WO 91/10725 (maize); Klein et al. (1988) Plant Physiol. 91:440-444 (maize); Fromm et al. (1990) Biotechnology 8:833-839; and Gordon-Kamm et al. (1990) Plant Cell 2:603-618 (maize); Hooydaas-Van Slogteren & Hooykaas (1984) Nature (London) 311:763-764; Bytebier et al. (1987) Proc. Natl. Acad. Sci. USA 84:5345-5349 (Liliaceae); De Wet et al. (1985) In The Experimental Manipulation of Ovule Tissues, ed. G. P. Chapman et al., pp. 197-209. Longman, N.Y. (pollen); Kaeppler et al. (1990) Plant Cell Reports 9:415-418; and Kaeppler et al. (1992) Theor. AppL. Genet 84:560-566 (whisker-mediated transformation); U.S. Pat. No. 5,693,512 (sonication); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750; Agrobacterium mediated maize transformation U.S. Pat. No. 5,981,840; silicon carbide whisker methods (Frame Br, Drayton P R, Bagnall S V, Lewnau J, Bullock W P, Wilson H M, Dunwell J M, Thompson J A and Wang K, 1994, Plant J. 6:941-948); laser methods (Guo Y, Liang, H and Berns M W, 1995, Physiologia Plantarum 93:19-24); sonication methods (Shiping Bao, Brian D. Thrall and Douglas L. Miller (1997) Ultrasound in Medicine & Biology 23:953-959; Finer K R, Finer J J (2000) Lett Appl Microbiol, 30:406-10; Amoah B K, Wu H, Sparks C, Jones H D, 2001 J Exp Bot 52:1135-42); polyethylene glycol mediated introduction of CONs in plant protoplasts (Krens R A, Molendijk L, Wullems G J and Schilperoort, R A, 1982, Nature 296:72-77); protoplasts of monocot and dicot cells can be transformed using electroporation (Fromm M, Taylor L P, Walbot V, 1985, Proc. Natl. Acad. Sci. USA 82:5824-5828) and microinjection (Crossway A, Oakes J V, Irvine J M, Ward B, Knauf V C and Shewmaker C K, 1986, Mol. Gen. Genet 202, 179-185); all of which are herein incorporated by reference.

The target for transformation could be in the form of plant cells, tissues, or organs such as embryo, callus, leaf, inflorescence, root, shoot or seed. In other methods plant gametes, microspores, pollen, mother cells, zygote, or nucellar cells can be used. Such genomic modifications using cationic oligonucleotides can also be performed in subcellular organelles such as chloroplasts and mitochondria.

Plants cells transformed with a plant expression vector can be regenerated, e.g., from single cells, callus tissue or leaf discs according to standard plant tissue culture techniques. Various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch et al., Science 227:1229-1231 (1985) and Fraley et al., Proc. Natl. Acad. Sci. U.S.A. 80:4803 (1983). This procedure typically produces shoots within two to four weeks and these transformant shoots are then transferred to an appropriate root-inducing medium containing the, selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee et al., Ann. Rev. of Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see generally, The Maize Handbook, Freeling and Walbot, Eds., Springer, New York (1994); Corn and Corn Improvement, 3_{rd} edition, Sprague and Dudley Eds., American Society of Agronomy, Madison, Wis. (1988).

The cells which have been altered by the methods of the invention may also be grown into plants in accordance with conventional ways. See, for example, McCormick et al. (1986) Plant Cell Reports 5:81-84 and Gruber et.al., 1993, “Vectors for Plant Transformation” In: Methods in Plant Molecular Biology and Biotechnology; Glick and Thompson, eds., CRC Press, Inc., Boca Raton, pages 89-119; Gordon-Kamm et al., The Plant Cell, 2:603-618 (1990); . These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited.

The following examples are offered by way of illustration and not by way of limitation.

### EXAMPLES

### Example 1

Synthesis of Cationic Oligonucleotides

Synthesis of cationic oligonucleotides of a specific sequence can be performed using methods reported earlier [Dagle et al. (1990) Nucleic Acid Research vol. 18, 4751-4757; Dagle and Weeks (1996), Nucleic Acid Research vol.24, pp2143-2149; Weeks DL and Dagle J (1998) U.S. Pat. No. 5,734,040]. The actual sequence of a cationic oligonucleotide will depend on the sequence of the genomic target to be modified. The oligonucleotide sequence can be complementary to the “minus” or non-transcribed strand of the region of the gene to be modified or it can be complementary to the transcribed or “plus” strand of the region of the gene to be modified. The gene modification efficiency of the cationic oligonucleotides may be different for the transcribed (or “plus”) DNA strand than that for the non-transcribed (or “minus”) DNA strand. In addition to the genomic target sequence to be modified, another factor, which affects the composition of cationic oligonucleotides, will be the number of cationic phosphoramidate internucleoside linkages in a specific cationic oligonucleotide molecule. Thus, a specific cationic oligonucleotide may contain one or more cationic phosphoramidite internucleoside linkages such than the total amount of cationic phosphoramidite internucleoside linkages in a specific cationic oligonucleotide may range up to 100%. Empirical determination of optimal conditions for specific applications of the cationic oligonucleotides can readily be determined.

### Example 2

Introduction of Cationic Oligonucleotides into Plant Cells

Described in this example are methods one may use for targeted modification of the genome of a plant cell.

Maize Particle-Mediated DNA Delivery

An appropriate cationic oligonucleotide can be introduced into maize cells capable of growth on suitable maize culture medium. Such competent cells can be from maize suspension culture, callus culture on solid medium, freshly isolated immature embryos or meristem cells. Immature embryos of the Hi-II genotype can be used as the target cells. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Musashige and Skoog, 1962, Physiol. Plant 15:473-497, with 0.25 M sorbitol). The embryos on the high-osmotic medium are used as the bombardment target, and are left on this medium for an additional 18 hours after bombardment.

For particle bombardment, plasmid DNA (described above) is precipitated onto 1.8 μm tungsten particles using standard CaCl₂- spermidine chemistry (see, for example, Klein et al., 1987, Nature 327:70-73). Each plate is bombarded once at 600 PSI, using a DuPont Helium Gun (Lowe et al., 1995, Bio/Technol 13:677-682). For typical media formulations used for maize immature embryo isolation, callus initiation, callus proliferation and regeneration of plants, see Armstrong, C., 1994, In “The Maize Handbook”, M. Freeling and V. Walbot, eds. Springer Verlag, N.Y., pp 663-671.

Within 1-7 days after particle bombardment, the embryos are moved onto N6-based culture medium containing 3 mg/l of the selective agent bialaphos. Embryos, and later callus, are transferred to fresh selection plates every 2 weeks. The calli developing from the immature embryos are screened for the desired phenotype. After 6-8 weeks, transformed calli are recovered.

Soybean Transformation

Soybean embryogenic suspension cultures are maintained in 35 ml liquid media SB196 or SB172 in 250 ml Erlenmeyer flasks on a rotary shaker, 150 rpm, 26C with cool white fluorescent lights on 16:8 hr day/night photoperiod at light intensity of 30-35 uE/m2s. Cultures are subcultured every two weeks by inoculating approximately 35 mg of tissue into 35 ml of fresh liquid media. Alternatively, cultures are initiated and maintained in 6-well Costar plates.

SB 172 media is prepared as follows: (per liter), 1 bottle Murashige and Skoog Medium (Duchefa # M 0240), 1 ml B5 vitamins 1000× stock, 1 ml 2,4-D stock (Gibco 11215-019), 60 g sucrose, 2 g MES, 0.667 g L-Asparagine anhydrous (GibcoBRL 11013-026), pH 5.7. SB 196 media is prepared as follows: (per liter) 10 ml MS FeEDTA, 10 ml MS Sulfate, 10 ml FN-Lite Halides, 10 ml FN-Lite P,B,Mo, 1 ml B5 vitamins 1000× stock, 1 ml 2,4-D, (Gibco 11215-019), 2.83 g KNO3, 0.463 g (NH4)2SO4, 2 g MES, 1 g Asparagine Anhydrous, Powder (Gibco 11013-026), 10 g Sucrose, pH 5.8. 2,4-D stock concentration 10 mg/ml is prepared as follows: 2,4-D is solubilized in 0.1 N NaOH, filter-sterilized, and stored at −20° C. B5 vitamins 1000× stock is prepared as follows: (per 100 ml)—store aliquots at −20° C., 10 g myo-inositol, 100 mg nicotinic acid, 100 mg pyridoxine HCI, 1 g thiamin.

Soybean embryogenic suspension cultures are transformed with various plasmids by the method of particle gun bombardment (Klein et al., 1987; Nature, 327:70. To prepare tissue for bombardment, approximately two flasks of suspension culture tissue that has had approximately 1 to 2 weeks to recover since its most recent subculture is placed in a sterile 60×20 mm petri dish containing 1 sterile filter paper in the bottom to help absorb moisture. Tissue (i.e. suspension clusters approximately 3-5 mm in size) is spread evenly across each petri plate. Residual liquid is removed from the tissue with a pipette, or allowed to evaporate to remove excess moisture prior to bombardment. Per experiment, 4-6 plates of tissue are bombarded. Each plate is made from two flasks.

To prepare gold particles for bombardment, 30 mg gold is washed in ethanol, centrifuged and resuspended in 0.5 ml of sterile water. For each plasmid combination (treatments) to be used for bombardment, a separate micro-centrifuge tube is prepared, starting with 50 μl of the gold particles prepared above. Into each tube, the following are also added; 5 μl of plasmid DNA (at 1 μg/μl), 50 μl CaCl2, and 20 μl 0.1 M spermidine. This mixture is agitated on a vortex shaker for 3 minutes, and then centrifuged using a microcentrifuge set at 14,000 RPM for 10 seconds. The supernatant is decanted and the gold particles with attached, precipitated DNA are washed twice with 400 μl aliquots of ethanol (with a brief centrifugation as above between each washing). The final volume of 100% ethanol per each tube is adjusted to 40 μl, and this particle/DNA suspension is kept on ice until being used for bombardment.

Immediately before applying the particle/DNA suspension, the tube is briefly dipped into a sonicator bath to disperse the particles, and then 5 UL of DNA prep is pipetted onto each flying disk and allowed to dry. The flying disk is then placed into the DuPont Biolistics PDS1000/HE. Using the DuPont Biolistic PDS1000/HE instrument for particle-mediated DNA delivery into soybean suspension clusters, the following settings are used. The membrane rupture pressure is 1100 psi. The chamber is evacuated to a vacuum of 27-28 inches of mercury. The tissue is placed approximately 3.5 inches from the retaining/stopping screen (3rd shelf from the bottom). Each plate is bombarded twice, and the tissue clusters are rearranged using a sterile spatula between shots.

Following bombardment, the tissue is re-suspended in liquid culture medium, each plate being divided between 2 flasks with fresh SB196 or SB172 media and cultured as described above. Four to seven days post-bombardment, the medium is replaced with fresh medium containing a selection agent. The selection media is refreshed weekly for 4 weeks and once again at 6 weeks. Weekly replacement after 4 weeks may be necessary if cell density and media turbidity is high.

Four to eight weeks post-bombardment, green, transformed tissue may be observed growing from untransformed, necrotic embryogenic clusters. Isolated, green tissue is removed and inoculated into 6-well microtiter plates with liquid medium to generate clonally-propagated, transformed embryogenic suspension cultures.

Each embryogenic cluster is placed into one well of a Costar 6-well plate with 5 mis fresh SB196 media with selection agent. Cultures are maintained for 2-6 weeks with fresh media changes every 2 weeks. When enough tissue is available, a portion of surviving transformed clones are subcultured to a second 6-well plate as a back-up to protect against contamination.

To promote in vitro maturation, transformed embryogenic clusters are removed from liquid SB196 and placed on solid agar media, SB 166, for 2 weeks. Tissue clumps of 2-4 mm size are plated at a tissue density of 10 to 15 clusters per plate. Plates are incubated in diffuse, low light (<10 μE) at 26+/−1° C. After two weeks, clusters are subcultured to SB 103 media for 3-4 weeks.

SB 166 is prepared as follows: (per liter), 1 pkg. MS salts (Gibco/BRL-Cat# 11117-017), 1 ml B5 vitamins 1000× stock, 60 g maltose, 750 mg MgCl2 hexahydrate, 5 g activated charcoal, pH 5.7, 2 g gelrite. SB 103 media is prepared as follows: (per liter), 1 pkg. MS salts (Gibco/BRL-Cat# 11117-017), 1 ml B5 vitamins 1000× stock, 60 g maltose, 750 mg MgCl2 hexahydrate, pH 5.7, 2 g gelrite. After 5-6 week maturation, individual embryos are desiccated by placing embryos into a 100×15 petri dish with a 1 cm 2 portion of the SB103 media to create a chamber with enough humidity to promote partial desiccation, but not death.

Approximately 25 embryos are desiccated per plate. Plates are sealed with several layers of parafilm and again are placed in a lower light condition. The duration of the desiccation step is best determined empirically, and depends on size and quantity of embryos placed per plate. For example, small embryos or few embryos/plate require a shorter drying period, while large embryos or many embryos/plate require a longer drying period. It is best to check on the embryos after about 3 days, but proper desiccation will most likely take 5 to 7 days. Embryos will decrease in size during this process.

Desiccated embryos are planted in SB 71-1 or MSO medium where they are left to germinate under the same culture conditions described for the suspension cultures. When the plantlets have two fully-expanded trifoliate leaves, germinated and rooted embryos are transferred to sterile soil and watered with MS fertilizer. Plants are grown to maturity for seed collection and analysis. Embryogenic cultures from the CycE treatment are expected to regenerate easily. Healthy, fertile transgenic plants are grown in the greenhouse. Seed-set on CycE transgenic plants is expected to be similar to control plants, and transgenic progeny are recovered.

SB 71-1 is prepared as follows: 1 bottle Gamborg's B5 salts w/sucrose (Gibco/BRL-Cat# 21153-036), 10 g sucrose, 750 mg MgCl2 hexahydrate, pH 5.7, 2 g gelrite. MSO media is prepared as follows: 1 pkg Murashige and Skoog salts (Gibco 11117-066), 1 ml B5 vitamins 1000× stock, 30 g sucrose, pH 5.8, 2 g Gelrite.

### Example 3

Cationic Oligonucleotide-Mediated Genomic Modifications of a Marker Gene

The jellyfish green fluorescent protein (GFP) has been used as a vital reporter in plants (Chiu W, Niwa Y, Zeng W, Hirano T, Kobayashi H and Sheen J, 1995, Curr Biol. 6:325-330) including corn (Zhu T, Peterson D J, Tagliani L, St Clair G, Baszczynski C L, Bowen B, 1999, Proc Natl Acad Sci USA 96:8768-8773). We have generated maize callus lines, which contain stably integrated single copy of a mutant form of GFP. Codon no. three of the mutant gene has a single base substitution (AAG to TAG) causing a premature termination of translation resulting in GFP-phenotype (Table 1).

| Construct | GFP Sequence | Phenotype |
|---|---|---|
| Ubi-GEP | SEQ ID NO: 1 | GFP+ |
| | ATGTCCAAGGGC... | |
| | METSERLYSGLY... | |
| Ubi-GFPm | SEQ ID NO: 2 | GFP− |
| | ATGTCCTAGGGC | |
| | METSERSTOP | |

Cationic oligonucleotide-mediated genomic modification of Ubi-GFPm for targeted conversion of TAG to MG results in restoration of the GFP+phenotype. Sequences of cationic oligonucleotides (CON) useful for such a conversion are shown below in 1 (A) and 1 (B). The cationic oligonucleotide sequence shown in 1 (A) is complementary to the transcribed strand (TransTarget) of the mutated GFP transgene. In another embodiment, the cationic oligonucleotide sequence complementary to the non-transcribed strand of the transgene 1 (B) can be used to introduce the desired mutation. Cationic oligonucleotides with differing % of positively charged internucleoside linkages up to 100% are used for such modifications.

Cationic Oligonucleotide Sequence for Modification of GFPm

| | |
|---|---|
| 1(A) | |
| 5′tctagaggatccaacaATGTCCTAGGGCGAGGAGCTCTTCACCGGCGTGGTGCCC3′- | SEQ ID NO: 3 |
| TransTarget | |
| 5′agatctcctaggttgtTACAGGTTCCCGCTCCTCCTCTTCACCGGCGTGGTGCCC3′- | SEQ ID NO: 4 |
| CON | |
| 1(B) | |
| 3′agatctcctaggttgtTACACGATCCCGCTCCTCCTCTTCACCGGCGTGGTGCCC5′- | SEQ ID NO: 5 |
| NonTransTarget | |
| 3′tctagaggatccaacaATGTCCTAGGGCGAGGAGCTCTTCACCGGCGTGGTGCCC5′- | SEQ ID NO: 6 |
| CON | |

A. TransTarget: Nucleotide sequences of the transcribed strand of GFPm surrounding the translation start codon (ATG). Cationic oligonucleotide:

corresponding cationic oligonucleotide for modifying the TAG stop codon to AAG (Lys).

B. NonTransTarget: Nucleotide sequence of the non-transcribed strand of GFPm surrounding the translation start codon (TAC).

Cationic oligonucleotide: Corresponding cationic oligonucleotide for modifying the stop codon of the transcribed strand.

Position of the positively charged internucleoside linkages is underlined.

### Example 4

Cationic Oligonucleotide-Mediated Genomic Modifications for Herbicide Resistance

Resistance of plants to synthetic or naturally occurring herbicides is important for many plant breeding and food production goals. This example describes use of cationic oligonucleotides for introducing two specific, heritable modifications in the maize acetohydroxyacid synthase (AHAS) gene. The first modification is a single base substitution from TCA to TGA resulting in a Ser→Asn mutation at amino acid position 621 for conferring resistance to imidazoline compounds. The second modification is at amino acid number 165 (Pro→Ala) conferring resistance to sulfonylurea compounds. As described in Example 3 above, the oligonucleotides may be designed to modify either the transcribed or the non-transcribed strand of the gene and the extent of cationic modifications of the internucleotide linkages of the cationic oligonucleotides may vary up to 100%.

| | | |
|---|---|---|
| 2(A)Ser621-Asn | | |
| 3′GTACACAACGGATACTAGGGATCACCACCCCGAAAGTTCTC5′ | Transcribed strand | SEQ ID NO: 7 |
| Ser | | |
| 5′CATGTGTTGCCTATGATCCCTATTGGTGGGGCTTTCAAGAG3′ | CON-1 | SEQ ID NO: 8 |
| Asp | | |
| 5′CATGTGTTGCCTATGATCCCTAGTGGTGGGGCTTTCAAGAG3′ | Non-Transcribed | SEQ ID NO: 9 |
| | Strand | |
| 3′GTACACAACGGATACTAGGGATTACCACCCCGAAAGTTCTG5′ | CON-2 | SEQ ID NO: 10 |
| 2(B)Pro106-Ala | | |
| 3′CAGCCGTAGTGCCCTGTCCACGGCGCTGCGTACTAACCG5′ | Transcribed Strand | SEQ ID NO: 11 |
| Pro | | |
| 5′GTCGCCATCACGGGACAGGTGGCGCGACGCATGATTGGC3′ | Con-1 | SEQ ID NO: 12 |
| Ala | | |
| 5′GTCGCCATCACGGGACAGGTGGCGCGACGCATGATTGGC3′ | | SEQ ID NO: 13 |
| 3′CAGCGGTAGTGCCCTGTCCACGGCGCTGCGTACTAACCG5′ | Con-2 | SEQ ID NO: 14 |

### Example 5

Cationic Oligonucleotide-Mediated Genomic Modifications for Disease Resistance

Plants have evolved very complex and sophisticated defense mechanisms against invading pathogens. These include production of anti-microbial compounds, oxidative bursts, lignin formation, expression of a number of pathogenesis related genes, as well as infection-induced localized cell-death (Talbot N J, Trends Microbiol. 1995, 3:9-16; Howard R J, Valent B, Annu. Rev. Microbiol. 1996, 50:491-512; Ronald P C, Plant Mol. Biol. 1997, 35:179-186; Hamer J E, Talbot N J Curr. Opin. Microbiol 1998, 1:693-697). These defense response mechanisms are usually activated by the interaction of a resistance gene (termed R gene) in the host and an avirulance gene (AVR gene) in the pathogen (Talbot N J, Trends Microbiol. 1995, 3:9-16; Howard R J, Valent B, Annu. Rev. Microbiol. 1996, 50:491-512; Ronald P C, Plant Mol. Biol. 1997, 35:179-86; Hamer J E, Talbot N J Curr. Opin. Microbiol 1998,1:693-697). Often, this gene-for-gene interaction is very specific for the host/pathogen pair and is believed to operate via a receptor-ligand type of interaction. Thus, the R gene product acts as a receptor, which recognizes and binds a ligand or elicitor, produced directly or indirectly by the AVR gene from the pathogen. This specific interaction is then believed to activate the defense response (Talbot N J, Trends Microbiol. 1995, 3:9-16; Howard R J, Valent B, Annu. Rev. Microbiol. 1996, 50:491-512; Ronald P C, Plant Mol. Biol. 1997, 35:179-186; Hamer J E, Talbot N J Curr. Opin. Microbiol 1998,1:693-697)

Analysis of R genes and their cognate protein products clearly indicates presence of common structural motifs such as leucine rich-repeats (LRR) that may be important in their interactions with the ligands. On the contrary, AVR genes show little or no structural similarities. Physical interactions between the R gene products and respective AVR gene products have been established in a number of host-pathogen pairs using genetic and biochemical methods. One such pair of interacting R and AVR genes is evident in rice and its fungal pathogen Magnaporthe grisea. The resultant disease, rice blast, is one of the most devastating plant diseases worldwide. Rice blast causes between 11% and 30% crop losses annually. This represents a loss of 157 million tones of rice. One way of reducing these losses is to plant rice varieties resistant to the fungus. Extensive research is being done on understanding the molecular mechanism underlying the observed resistance of rice varieties to the pathogen M. grisea. These studies have revealed the presence of the rice R gene Pi-ta and the Avr gene Avr-Pita as the gene pair responsible for the rice blast disease. (Jia Y, McAdams S A, Bryan G T, Hershey H P, Valent B, EMBO J, 2000, 19:4004-4014) Furthermore, this group has clearly shown that a single amino acid difference distinguishes resistant and susceptible alleles of the rice blast resistance gene Pi-ta (Bryan G T, Wu K S, Farrall L, Jia Y, Hershey H P, McAdams S A, Faulk K N, Donaldson G K, Tarchini R, Valent B, Plant Cell, 2000 12:2033-2046). Thus the susceptible varieties have a serine at position 918 whereas the resistant varieties have an alanine at the same position (See A below). This observation provides us a unique opportunity to create blast resistant rice varieties using the method of the present invention.

A

SEQ ID NO: 15

| | |
|---|---|
| TTACCTTCTATGCATCTTCAACCTGACTTGATGATTGTT* | Sensitive |
| Ser | |

SEQ ID NO: 16

| | |
|---|---|
| TTACCTGCTATGCATCTTCAACCTGACTTGATGATTGTT* | Resistant |
| Ala | |

Specifically, following sets of oligonucleotides will be used to generate a single base change at the codon no. 918 of the rice Pi-ta gene to convert the susceptible Ser to the resistant Ala:

| | |
|---|---|
| 5′CCGTGGCTTCTATCTTTACCTGCTATGCATCTTCAACCTGACTTGATGATTGTT3′ | SEQ ID NO: 17 |
| Con-1 | |
| 3′GGCACCGAAGATAGAAATGGACGATACGTAGAAGTTGGACTGAACTACTAACAA5′ | SEQ ID NO: 18 |
| Con-2 | |

### Example 6

Cationic Oligonucleotide-Mediated Genomic Modifications for Improving Agronomic Traits

Modulation of Lignin Composition by Cationic Oligonucleotide Induced Genomic Modification (CONIGM)

Background: Lignin is a very complex and highly heterogeneous biopolymer present in all vascular plants. It is composed of a hydrophobic network of one or more of three basic types of units called monolignols. These three monolignols, p-coumaric, coniferyl and synapyl alcohols, are products of the phenylpropanoide pathway. Extensive studies have been carried out on the enzymology and regulatory mechanisms of lignin biosynthesis (reviewed in Baucher et al., 1998; Whetten et al., 1998 and Anterola & Lewis, 2002). Interestingly, while a number of plant enzymes are involved in lignin formation, there are no known plant enzymes involved in degradation of this complex biomolecule.

The structural heterogeneity and physico-chemical complexity of lignin are of great economic consequence. For example, in the paper industry, removal of lignins requires extensive treatments such as chemical pulping (using the widely used Kraft method), which are expensive, time consuming and damaging to the environment. On the other hand, the feed industry faces another set of challenges due to the complex physico-chemical nature of lignins. In nature, lignins are closely associated with the cell wall polysaccharides of the silage and thereby interfere with digestion of carbohydrates by limiting their accessibility to the cell-wall hydrolyzing enzymes in the ruminant stomach, thereby reducing the nutritional value of the feed. Therefore, there is considerable interest in reducing the chemical complexity of lignins using biotechnological approaches.

The high degree of heterogeneity and complexity can also be viewed as an advantage vis-à-vis the manipulation of lignin biosynthesis using biotechnological methods. For example, numerous plant mutants with variations in composition and/or quantity of lignin have been reported, indicating remarkable plasticity within the pathways of lignin biosynthesis. Some of these mutant plants appear to survive well despite such significant variation in the composition/and or quantity of lignin. This allows one to design methods for modulating specific enzymatic steps involved in the phenylpropanoid pathway in order to regulate lignin quality and/or quantity. Most of the genetic engineering approaches for modifying lignin content and/or composition have utilized the ‘antisense inhibition’ or sense co-suppression’ of target genes involved in lignin biosynthesis. Both methods require generation of transgenic plants via traditional methods such as particle bombardment and/or Agrobacterium infection and suffer from drawbacks associated with these methods. We propose a novel method of modulating lignin content/composition via targeted gene modification through cationic oligonucleotides. This method utilizes cationic oligonucleotides, which are designed to specifically hybridize with a defined region of the target gene to introduce a desired change in the nucleotide sequence of the target gene. The targeted modification takes place in vivo utilizing cellular DNA repair pathways.

The present example describes a method for introducing targeted modifications in a lignin biosynthetic pathway gene to modulate its activity in such a way as to modify the quantity and/or composition of lignin in monocots such as maize, sorghum, rice, barley, oats and wheat as well as dicots such as soybean, sunflower, and alfalfa. Specifically, we will introduce targeted modification in the Ferulate-5-hydroxylase gene to modulate amount or activity of the enzyme, which in turn will modulate the quantity or quality of lignin produced in the said plant. Ferulate-5-hydroxylase (F5H) is the enzyme responsible for introducing hydroxyl group at the C5 position of ferulic acid. This enzyme is a monooxygenase found in the microsomal fraction and is associated with the Cytochrome P-450 (Grand, 1984). Recently, Meyer et al (PNAS, 1998) have cloned a cDNA for F5h from A. thaliana. Furthermore, it has been shown that a mutation in F5H gene of A. thaliana causes a significant reduction in the activity of this enzyme, which in turn results in modification of the lignin composition of the mutant plants (Marita et al., 1999 and Frank et al., 2000. The present example exploits these observations to introduce a targeted modification in the F5H gene via cationic oligonuclotides.

### References

1. Baucher M, Monties B, Van Montagu M and Boerjan W (1998) Crit Rev Plant Sci 7: 125-197

2. Whetten R W, MacKay J J and Sederoff R R (1998) Ann Rev Plant Mol Biol 49: 585-609

3. Anterola A M, Lewis N G Phytochemistry (2002) 61:221-94

4. Grand C (1984) FEBS Lett 169: 7-11

5. Meyer K, Shirley A M, Cusumano J C, Bell-Lelong B and Chappel C (1998) Proc Natl Acad Sci USA 95 :6619-6623

6. Marita J M, Ralph J, Haffield R D and Chappel C Proc Natl Acad Sci USA (1999) 96: 12328-12332

7. Franke R, McMichael C M, Meyer K, Shirley A M, Cusumano J C and Chapple C Plant J. (2000) 22 : 223-234

Modulation of F5H for Changes in Lignin Composition

| | | |
|---|---|---|
| 5′AAGCTGTTCAGCCGGCGCCGCGCCGAGACGTGGGTGGCCGTGCGCGACGAGTGCGCGGCG3′ | Trans | SEQ ID NO: 19 |
| Try | | |
| 5′AAGCTGTTCAGCCGGCGCCGCGCCGAGACGTGAGTGGCCGTGCGCGACGAGTGCGCGGCG3′ | CON-1 | SEQ ID NO: 20 |
| Stop | | |
| 3′TTCGACAAGTCGGCCGCGGCGCGGCTCTGCACCCACCGGCACGCGCTGCTCACGCGCCGC5′ | Non-Trans | SEQ ID NO: 21 |
| 3′TTCGACAAGTCGGCCGCGGCGCGGCTCTGCACTCACCGGCACGCGCTGCTCACGCGCCGC5′ | CON-2 | SEQ ID NO: 22 |

### Example 7

Modification of a Transgene

This example depicts the utility of cationic oligonulceotides for modification of a transgene introduced into the maize genome. Agrobacterium mediated transformation as substantially described in U.S. Pat. No. 5,981,840 was used to introduce a single copy of the plant transcription unit UBI::MOPAT::TAG::GFP::PINII into GS3 callus. In this construct, MOPAT is used as a selectable marker driven by the UBI promoter. Those skilled in the art will recognize that the presence of the stop codon TAG strategically placed at the end of UBI::MOPAT and at the beginning of the GFP coding region will preclude translation of the GFP protein. Such maize plants and tissues produced will be referred to as the target lines or target tissues. Those skilled in the art will also recognize that modification of this stop codon TAG to TAC will permit faithful translation of the entire transcriptional unit, thereby allowing expression of GFP. This was achieved using cationic oligonulceotides specifically designed to institute this modification. Table 1 shows the sequence and extent of modification of the cationic oligonulceotides used in this experiment. These oligonucleotides have been designed to correct the codon TAG to TAC, thereby changing it from a stop codon to a codon for the amino acid Tyrosine (Tyr). Oligonucleotides GMCOPHP 12 and 13 are complementary to the transcribed strand and GMCOPHP 17 and 18 are complementary to the non-transcribed strand. The cationic (diethylethylenediamine or DEED) modification of phosphodiester bonds in each oligonucleotide is highlighted in Table 1.

These oligonucleotides were introduced into the callus tissue and/or 10DAP embryos of four different GFP target lines using the biolistic gun method as described above. Prior to bombarding, the plates containing callus and/or embryos were placed in a sterile hood and irradiated with ultra-violet light (152-354 microj/cm²). This was followed by microprojectile bombardment using Bio-Red PDS1000-He particle delivery system (Zhu et al., 1996). For microprojectile bombardment, the cationic oligonucleotides (0.1 μg/μl) were treated with gold particles (60 μg/μl) and TFX50 (5 ul of TFX-50 for 1.0 μg of DNA) and finally resuspended in 100% ethanol.

The bombarded plates containing callus cultures or embryos were screened using Leica DC200 microscope at least twice between day 1 and 10 after bombarding. The GFP2 filter that was fitted to this microscope helped to view the positives or the callus cells that showed activation of GFP. Digital images Were captured and processed with Leica DC viewer.

Preliminary results indicate that cationic oligos are capable of making the correction that activates GFP. This system seems to work more efficiently in callus tissues than in embryos (data not shown). We also found that cationic oligos complementary to either transcribed or non-transcribed DNA strand are capable of making specific conversions. Our experiments consisted of treatments that ranged from A through G, as described in Table 2. Treatment A though D, describes the set-up using different cationic oligos, and their delivery into the target lines. Treatment E is a negative control, where neither a plasmid nor an oligo was bombarded into GS3 callus. Construct PHP7921 contains a GFP gene driven by Ubiquitin promoter. In treatment F, GS3 callus were bombarded with PHP7921 (as a positive control), to transiently assay if the conditions provided in the experiment were favorable for efficient particle delivery. We also included another treatment G, where the callus from a stably transformed line (Agrobacterium mediated) of PHP 17228 was used as the base material. In PHP 17228, Ubiquitin promoter drives MOPAT followed by functional GFP, without the TAG stop codon. Treatment G was included to compare and monitor the stable expression of GFP in PHP 17228 callus material to induced GFP activation under similar biolistic/culture conditions. The details of screening and positives obtained in four different experiments are presented in Tables 3 through 6. These converted events have been picked and are being monitored for growth and GFP fluorescence.

[image]

TABLE 2
CONIGM Experimental set up

| | Oligo/ | | DNA | | | |
|---|---|---|---|---|---|---|
| Treat- | Plasmid | | (0.1 | Sel | | |
| ment | (0.1 ug/ul) | Loading | ug/ul) | media | Pressure | Description |
| A | GMOPHP12 | TFX-50 | used | 560 R | 650 psi | Experiment |
| B | GMOPHP13 | TFX-50 | used | 560 R | 650 psi | Experiment |
| C | GMOPHP17 | TFX-50 | used | 560 R | 650 psi | Experiment |
| D | GMOPHP18 | TFX-50 | used | 560 R | 650 psi | Experiment |
| E | | TFX-50 | not | 560 R | 650 psi | Control +ve |
| | | | used | | | |
| F | PHP 7921 | TFX-50 | used | 560 R | 650 psi | Control +ve |
| G | | TFX-50 | not | 560 R | 650 psi | Control +ve |
| | | | used | | | |

TABLE 3
CONIGM Experiment 1

| | | 2nd Round No. of picks in each plate | | | | | |
|---|---|---|---|---|---|---|---|
| To material | Screen Date | Plate 1 | Plate 2 | Plate 3 | Plate 4 | Plate 5 | Comments |
| PHP 11207 #6-1 | A | 0 | 1 | 0 | 0 | 0 | |
| | B | 0 | 0 | 0 | 0 | 0 | |
| | C | 0 | 0 | 0 | 0 | 0 | |
| | D | 0 | 0 | 0 | 0 | 0 | |
| PHP 11207 #9-5 | A | 0 | 0 | 1 | 0 | 0 | |
| | B | 2 | 0 | 0 | 0 | 0 | |
| | C | 0 | 0 | 0 | 0 | 0 | |
| | D | 2 | 0 | 0 | 0 | 0 | |
| GS3 callus | E | negative | negative | negative | negative | negative | Control −ve |
| | F | several | several | several | several | several | Control +ve |
| | | spots | spots | spots | spots | spots | |
| PHP 17228 #2-10 | G | positive | positive | positive | positive | positive | Control +ve |

Several spots: refers to transient activity of PHP7921, which indicated that the experimental set up was good.
Positive: refers to the stable expression of the stable transformed GS3 line carrying the construct PHP 17228

TABLE 4
CONIGM Experiment 2

| | | | | Number of picks in each plate | | | | |
|---|---|---|---|---|---|---|---|---|
| To material | Treatment | Transferred | Screen Date | Plate 1 | Plate 2 | Plate 3 | Plate 4 | Plate 5 |
| PHP 11207 #1-11 | A | Sep. 18, 2001 | Sep. 19, 2002 | 0 | 0 | 0 | 0 | 0 |
| | B | | | 0 | 0 | 0 | 0 | 0 |
| PHP 11207 #1-12 | C | | | 0 | 3 | 0 | 0 | 0 |
| | D | | | 0 | 0 | 0 | 0 | 0 |
| PHP 17228 #2-10 | G | | | positive | positive | positive | positive | positive |

Positive: refers to the stable expression of the stable transformed GS3 line carrying the construct PHP17228

TABLE 5
CONIGM Experiment 3

| | | | | Number of picks in each plate | | | | |
|---|---|---|---|---|---|---|---|---|
| To material | Treatment | Transferred | Screen Date | Plate 1 | Plate 2 | Plate 3 | Plate 4 | Plate 5 |
| PHP 11207 #1-12 | A | | Oct. 14, 2002 | 0 | 7 | 1 | 2 | |
| PHP 11207 #1-12 | B | | Oct. 15, 2002 | 5 | 11 | 2 | 1 | 2 |
| PHP 11207 #1-12 | C | | Oct. 15, 2002 | 3 | | | | |
| PHP 11207 #1-11 | A | | Oct. 15, 2002 | 0 | | | | |
| PHP 11207 #1-11 | C | | Oct. 15, 2002 | 0 | 0 | 0 | 1? | |
| PHP 11207 #1-11 | D | | Oct. 15, 2002 | 1? | 1? | 0 | 0 | 0 |
| PHP 17228 #2-10 | G | | Oct. 15, 2002 | positive | positive | positive | positive | positive |

**Refers to plates lost due to contamination
?Refers to a signal that is distinguishable from background, but indefinite
Positive: refers to the stable expression of the stable transformed GS3 line carrying the construct PHP 17228

TABLE 6
CONIGM Experiment 4

| To material | Screen Date | Plate 1 | Plate 2 | Plate 3 | Plate 4 | Plate 5 | Comments |
|---|---|---|---|---|---|---|---|
| PHP 11207 #6-1 | A | 2 | 0 | 0 | ** | ** | |
| | B | 0 | ** | ** | ** | ** | |
| | C | 2 | 0 | 0 | 0 | contamin | |
| | D | 0 | 0 | 0 | 2 | 0 | |
| PHP 11207 #9-5 | A | 2 | 4 | 0 | 5 + 3 = 8 | contamin | |
| | B | 0 | 0 | 0 | 0 | 0 | |
| | C | 0 | 0 | 1 + 1? | 0 | 0 | |
| | D | 0 | 0 | 0 | 0 | 1? | |
| GS3 callus | E | negative | negative | negative | ** | ** | Control −ve |
| | F | several | several | several | several | ** | Control +ve |
| | | spots | spots | spots | spots | | |

**Refers to plates lost due to Contamination
?Refers to a signal that is distinguishable from background, but indefinite
Several spots: refers to transient activity of PHP 7921, which indicates that the experimental set up was good.
Negative: refers to no detection of GFP fluorescence

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. A template oligonucleotide comprising regions complementary to plant genomic DNA, the complementary regions flank a non-complementary region, and the template oligonucleotide comprises a single-stranded nucleic acid comprising at least one cationic phosphoramidate internucleoside linkage.

2. The template oligonucleotide of claim 1, wherein the plant genomic DNA is a regulatory region.

3. The template oligonucleotide of claim 1, wherein the plant genomic DNA is a promoter region.

4. The template oligonucleotide of claim 1, wherein the plant genomic DNA is a from a junction of an intron and an exon.

5. The template oligonucleotide of claim 1, wherein the plant genomic DNA is a transformed DNA.

6. The template oligonucleotide of claim 5, wherein the plant genomic DNA is a recombination site.

7. The template oligonucleotide of claim 1, wherein the plant genomic DNA is a transposable element.

8. The template oligonucleotide of claim 1, wherein the at least one non-homologous nucleotide produces a stop codon.

9. The template oligonucleotide of claim 1, wherein the at least one non-homologous nucleotide produces a nucleotide deletion in the plant genome.

10. The template oligonucleotide of claim 1, wherein the plant genomic DNA encodes a protein.

11. The template oligonucleotide of claim 1, wherein the plant genomic DNA encodes a recombinase protein.

12. The template oligonucleotide of claim 1, wherein the plant genomic DNA is involved with herbicide tolerance in the plant.

13. The template oligonucleotide of claim 1, wherein the plant genomic DNA is involved with disease resistance in a plant.

14. A plant cell comprising a template oligonucleotide comprising regions complementary to plant genomic DNA, the complementary regions flank a non-complementary region, and the template oligonucleotide comprises a single-stranded nucleic acid comprising at least one cationic phosphoramidate internucleoside linkage.

15. A method of introducing a predetermined alteration in genomic DNA of a plant cell, the method comprising introducing a template oligonucleotide into the plant cell, the template oligonucleotide comprising regions complementary to the plant genomic DNA, the complementary regions flank a non-complementary region, and wherein the template oligonucleotide comprises a single-stranded nucleic acid comprising at least one cationic phosphoramidate internucleoside linkages.

16. The method of claim 15, wherein the plant genomic DNA is a regulatory region.

17. The method of claim 15, wherein the plant genomic DNA is a promoter region.

18. The method of claim 15, wherein the plant genomic DNA is from a junction of an intron and an exon.

19. The method of claim 15, wherein the plant genomic DNA is transformed DNA.

20. The method of claim 19, wherein the transformed DNA is a recombination site.

21. The method of claim 15, wherein the plant genomic DNA is involved with herbicide tolerance in the plant.

22. The method of claim 15, wherein the plant genomic DNA is involved with disease resistance in a plant.

23. The method of claim 15, wherein the at least one non-homologous nucleotide produces a stop codon.

24. The method of claim 15, wherein the at least one non-homologous nucleotide produces a nucleotide deletion in the plant genome.

25. The method of claim 15, wherein the plant genomic DNA encodes a protein.

26. The method of claim 15, wherein the protein is a recombinase protein.

27. The method of claim 15, wherein the predetermined alteration confers herbicide resistance to the plant.

28. A method to inactivate a polynucleotide of interest introduced into a plant genome, the method comprising introducing into a plant cell a template oligonucleotide capable of implementing a modification in the polynucleotide of interest such that the modification prevents expression of the polynucleotide of interest, wherein the template oligonucleotide comprises regions complementary to the polynucleotide of interest, the complementary regions flank a non-complementary region, and wherein the template comprises a single-stranded nucleic acid comprising at least one cationic phosphoramidate internucleoside linkage.

29. The method of claim 28, wherein the modification is in a regulatory region.

30. The method of claim 28, wherein the modification is in a promoter region.

31. The method of claim 28, wherein the modification is in a coding region.

32. The method of claim 28, wherein the template oligonucleotide introduces a frameshift in the coding region.

33. The method of claim 28, wherein the template oligonucleotide introduces a premature stop codon in the in the coding region.

34. The method of claim 28, wherein the polynucleotide of interest encodes a selectable and/or scorable marker.

35. The method of claim 28, wherein the polynucleotide of interest is involved with herbicide resistance.

36. The method of claim 28, wherein the polynucleotide of interest is involved with disease resistance.

37. A method for creating a predetermined nucleotide pair mismatch in a plant genomic DNA upon which endogenous mismatch repair mechanisms can operate to create a nucleotide alteration, the method comprising introducing a template oligonucleotide into the plant cell, the template oligonucleotide comprising regions complementary to the plant genomic DNA, the complementary regions flank a non-complementary region, and the template oligonucleotide comprises a single-stranded nucleic acid comprising at least one cationic phosphoramidate internucleoside linkage.

38. The method of claim 37, wherein the plant polynucleotide encodes an herbicide resistance gene.

39. The method of claim 37, wherein the polynucleotide of interest is involved with herbicide resistance.


SEQUENCE LISTING (26 sequences)

SEQ ID NO: 1 - DNA, 12 bp, Aequorea victoria
atgtccaagggc

SEQ ID NO: 2 - DNA, 12 bp, Aequorea victoria
atgtcctagggc

SEQ ID NO: 3 - DNA, 55 bp, Aequorea victoria
tctagaggatccaacaatgtcctagggcgaggagctcttcaccggcgtggtgccc

SEQ ID NO: 4 - DNA, 55 bp, Aequorea victoria
agatctcctaggttgttacaggttcccgctcctcctcttcaccggcgtggtgccc

SEQ ID NO: 5 - DNA, 55 bp, Aequorea victoria
agatctcctaggttgttacaggatcccgctcctcctcttcaccggcgtggtgccc

SEQ ID NO: 6 - DNA, 55 bp, Aequorea victoria; identical to SEQ ID NO: 3

SEQ ID NO: 7 - DNA, 41 bp, Zea mays
gtacacaacggatactagggatcaccaccccgaaagttctc

SEQ ID NO: 8 - DNA, 41 bp, Zea mays
catgtgttgcctatgatccctattggtggggctttcaagag

SEQ ID NO: 9 - DNA, 41 bp, Zea mays
catgtgttgcctatgatccctagtggtggggctttcaagag

SEQ ID NO: 10 - DNA, 41 bp, Zea mays
gtacacaacggatactagggattaccaccccgaaagttctg

SEQ ID NO: 11 - DNA, 39 bp, Zea mays
cagcggtagtgccctgtccacggcgctgcgtactaaccg

SEQ ID NO: 12 - DNA, 39 bp, Zea mays
gtcgccatcacgggacaggtggcgcgacgcatgattggc

SEQ ID NO: 13 - DNA, 39 bp, Zea mays; identical to SEQ ID NO: 12

SEQ ID NO: 14 - DNA, 39 bp, Zea mays; identical to SEQ ID NO: 11

SEQ ID NO: 15 - DNA, 39 bp, Oryza sativa
ttaccttctatgcatcttcaacctgacttgatgattgtt

SEQ ID NO: 16 - DNA, 39 bp, Oryza sativa
ttacctgctatgcatcttcaacctgacttgatgattgtt

SEQ ID NO: 17 - DNA, 54 bp, Oryza sativa
ccgtggcttctatctttacctgctatgcatcttcaacctgacttgatgattgtt

SEQ ID NO: 18 - DNA, 54 bp, Oryza sativa
ggcaccgaagatagaaatggacgatacgtagaagttggactgaactactaacaa

SEQ ID NO: 19 - DNA, 60 bp, Arabadopsis thaliana
aagctgttcagccggcgccgcgccgagacgtgggtggccgtgcgcgacgagtgcgcggcg

SEQ ID NO: 20 - DNA, 60 bp, Arabidopsis thaliana
aagctgttcagccggcgccgcgccgagacgtgagtggccgtgcgcgacgagtgcgcggcg

SEQ ID NO: 21 - DNA, 60 bp, Arabadopsis thaliana
ttcgacaagtcggccgcggcgcggctctgcacccaccggcacgcgctgctcacgcgccgc

SEQ ID NO: 22 - DNA, 60 bp, Arabadopsis thaliana
ttcgacaagtcggccgcggcgcggctctgcactcaccggcacgcgctgctcacgcgccgc

SEQ ID NO: 23 - DNA, 20 bp, Aequorea victoria
cgcagatctacgtaccatcg

SEQ ID NO: 24 - DNA, 20 bp, Aequorea victoria
cgcagatctacgtaccatcg

SEQ ID NO: 25 - DNA, 21 bp, Aequorea victoria
acgatggtacgtagatctgcg

SEQ ID NO: 26 - DNA, 21 bp, Aequorea victoria
acgatggtacgtagatctgcg